(19) United States
(12) Patent Application Publication
(10) Pub. No.: US 2020/0330985 A1
(43) Pub. Date: Oct. 22, 2020

(54) A BIOSENSOR SYSTEM FOR USE IN POINT-OF-CARE APPLICATIONS AND A METHOD FOR FABRICATING SAME

(71) Applicant: DUBLIN INSTITUTE OF TECHNOLOGY, Dublin (IE)

(72) Inventors: Mohamed Oubaha, Dublin (IE); Brendan Duffy, Kilcullen (IE)

(21) Appl. No.: 16/090,064

(22) PCT Filed: Mar. 31, 2017
(86) PCT No.: PCT/EP2017/057707

§ 371 Date: Sep. 28, 2018

(30) Foreign Application Priority Data
Mar. 31, 2016 (GB) 1605465.2

(51) Int. Cl. B01L 3/00; G01N 33/53

(52) U.S. Cl. CPC B01L 3/502707; G01N 33/5302; B01L 2200/12; B01L 2300/0864; B01L 2300/12; B01L 2300/0636

(57) ABSTRACT

A biosensor system for use in point-of-care applications and a method for fabricating same

The present invention provides a biosensor system for use in point-of-care, POC, applications. The invention comprises a microfluidic channel for circulating biomolecules and a sensor adapted to receive the microfluidic channel and detect a plurality of different biomarkers from the biomolecules. The microfluidic channel and the sensor are formed on a photoreactive hybrid organic-inorganic sol-gel, PHOIS, resin.

### FIELD

The present invention relates to biosensor systems. More particularly, the present invention relates to a biosensor system for use in point-of-care applications and a method for fabricating same.

### BACKGROUND

The majority of medical diagnostic testing takes place in centralised hospital laboratories. However, it will be appreciated that such testing is time intensive as well as requiring the utilization of large-scale and expensive equipment. The development of biosensors has made point-of-care (POC) testing on inexpensive disposable chips feasible in respect of some medical conditions. POC testing has many major advantages over conventional testing at a clinic. It enables a patient to benefit from earlier treatments, due to the faster decision making process afforded by the technology, in addition to fewer hospital visits. For example, for people with diabetes, the ability to self-monitor often leads to improved adherence to treatment and reduced incidence of complications, while patients undergoing anticoagulation treatment benefit from quicker treatment optimization. POC testing is also clearly beneficial in critical care settings, where immediate testing for biomarkers can significantly affect outcomes and survival rates. Such testing also results in financial benefits for a clinic, due to the reduced workload associated with a decreased number of patient visits.

One drawback with respect to existing POC technology is that the POC devices are only able to diagnose in respect of one parameter only. This is a major issue for many diagnostic situations. For example, routine early detection of diseases such as cancers and cardiac enzymes, as well as the monitoring of several analytes in parallel (such as glucose, cholesterol and triglycerides) requires multianalyte testing.

Most of the multianalyte biosensors which are in existence are based on optical transduction techniques, and in particular luminescence based optical biosensors. In this regard, a group at the Naval Research Laboratory (NRL) in Washington have developed a multianalyte array biosensor using evanescent wave excitation technology which has been used by the defence forces for detection of biowarfare threats. One drawback of this implementation lies in the use of polydimethylsiloxane channels for biosensor spot deposition, which results in large sensor spots that negatively impact signal-to-noise performance from a biological and detection standpoint. A Swiss company by the name of Zeptosens has also produced a proprietary planar waveguide technology which provides multiplexed, quantitative biomolecular interaction analysis with high sensitivity in a microarray format. However, this technology has been optimised for high sensitivity DNA and protein arrays, and as such is not suitable for the demands of POC markets, where a low cost platform is required.

Some limited work has been carried out on other biosensors utilizing electrochemical and thermal transduction techniques. For example, a multianalyte electrochemical biosensor which can conduct up to eight simultaneous analyses on one chip has been developed. However, this biosensor still suffers from the practical problems of electrochemical sensors.

The Journal of Sol-Gel Science and Technology, vol. 48, 2008, X. Zhang et al., “Fabrication of microfluidic devices using photopatternable hybrid sol-gel coatings” pp 143-147 discloses the fabrication of a microfluidic device by performing photolithography on a photopatternable hybrid sol-gel layer which has been spin coated onto either a silicon or a glass substrate. The sol-gel layer is formed with 3-methacryloxpropyltrimetoxysilane (MPTS) as the photosensitive component and zirconium propoxide as the property modifier. In order to enhance the UV light efficiency when the photolithography processing is being performed on a glass substrate, this document further teaches that a removable thin copper layer can be deposited on one side of the glass substrate prior to undertaking the photolithography process.

This journal publication further mentions that such a microfluidic device could be used in biological analysis systems. However, there is no teaching in this context that a sol-gel material would be suitable for use as a sensor in such a biosensing application.

The Journal of Materials Chemistry, vol. 14, 2004 X. Zhang et al., “Thick UV-patternable hybrid sol-gel films prepared by spin coating”, pp. 357-361 also discloses a microfluidic device fabricated from a photopatternable hybrid sol-gel layer which has been spin coated onto a substrate. The sol-gel layer is formed through the use of MPTS and zirconium propoxide, as well as methacrylic acid and a photoinitiator, while the substrate is disclosed as being one of silicon, silica-on-silicon, glass or a PC board.

However, as was the case with the previous journal publication, there is no teaching in this document that a sol-gel material could be used as a sensor.

International Patent Publication No. WO 2010/019969 discloses the entrapment of biomolecules within a sol gel material for the rapid identification of nucleic acid. Similarly, US Patent Publication No. US 2003/0180964 refers to the functionalisation of a sol gel material through the deposition of a sensing material on top of the sol-gel layer.

Thus, it is an object of the present invention to provide a method for fabricating a point-of-care biosensor system which overcomes at least one of the above problems.

### SUMMARY

According to a first aspect of the invention, there is provided, as set out in the appended claims, a biosensor system for use in point-of-care, POC, applications comprising:
a microfluidic channel for circulating biomolecules; and
a sensor adapted to receive the microfluidic channel and detect a plurality of different biomarkers from the biomolecules;
wherein the microfluidic channel and the sensor are formed on a photoreactive hybrid organic-inorganic sol-gel, PHOIS, resin.

Accordingly, the present invention provides a biosensor system where both the microfluidic channel and the sensor are provided by a sol-gel material.

This is in contrast to existing systems which incorporate a sol gel material. In such systems, while the sol gel material may provide a particular functionality in the system (such as the encapsulation of biomolecules within the sol-gel coating), the actual sensor is formed from a different material, which may be deposited as a sensing layer.

The PHOIS resin may be adapted to exhibit high surface energy and photopatternability.

As a result the PHOIS resin material of the invention provides for ease of rapid platform microfabrication and the efficient transfer of matter via capillary-driven fluid flow.

The PHOIS resin may be prepared from an individual organosilane.

The PHOIS resin may be prepared from a combination of multiple organosilanes.

The organosilanes may comprise one or more of: 3-(trimethoxysilyi)propylmethacrylate, 3-(triethoxysilyl)propylmethacrylate, vinyltrimethoxysilane and vinyltriethoxysilane.

By preparing the sol gel material from one or more of these organosilanes, the inherent characteristic chemistry of the developed sol-gel provides sensing capabilities. Thus, the sol gel alone is able to provide the sensor of the biosensor system, in addition to providing its microfluidic channel.

The PHOIS resin may be prepared from a combination of a single organosilane and a transition metal complex.

The transition metal complex may comprise one or more transition metal alkoxide precursors and one or more ligands.

The organosilanes may comprise one or more of: 3-tri methoxypropyltrimethoxysilane, 3-triethoxypropyltrimethoxysilane, 3-aminopropyltrimethoxysilane, 3-aminopropyltriethoxysilane, vinyltrimethoxysilane, vinyltriethoxysilane, 3-glycidyloxypropyltrimethoxysilane, trimethoxyphenylsilane, triethoxyphenylsilane, methyltrimethoxysilane and methyltriethoxysilane.

The transition metal alkoxide precursors may comprise one or more of: titanium, zirconium, niobium, tantalum and vanadium.

The ligands may comprise one or more of: acrylic acid, methacrylic acid, pivalic acid, acetone, acetyl acetone, acetic acid and maleic acid.

The PHOIS resin may be prepared from a combination of one or more organosilane precursors and multiple transition metal precursors, which has been chelated with a ligand containing a methacrylic or acrylic group.

One or more highly reactive groups with affinity to the target biomolecules may be incorporated into the PHOIS resin.

Covalent silane based linker layers containing one or more highly reactive groups may be attached to the PHOIS surface.

The highly reactive groups may comprise one or more of: amino, epoxy or acrylic group.

The biosensor system may further comprise a single optical source coupled to the sensor and adapted to excite target biomolecules to enable detection of the biomarkers by the sensor.

The sensor may be adapted to detect the biomarkers through the excitation of fluorescence from the target biomolecules by means of the optical source.

The sensor may comprise a plurality of sensor windows for detection of the plurality of different biomarkers, wherein each sensor window is adapted to detect a different biomarker.

The microfluidic channel may comprise a plurality of microfluidic channels, each microfluidic channel being coupled to a different sensor window.

One end of each microfluidic channel may be coupled to an inlet for receiving the biomolecules and the other end of each microfluidic channel is coupled to its associated sensor window.

The plurality of microfluidic channels may comprise multiple y-branch microfluidic channel splitters.

The biosensor system may further comprise a common wicking zone, wherein each sensor window is coupled to the wicking zone.

In another embodiment of the invention a method is provided for fabricating a biosensor system for use in point-of-care, POC, applications, the biosensor system comprising a microfluidic channel and a sensor, the method comprising the step of:
forming the microfluidic channel and forming the sensor from photolithography of a photoreactive hybrid organic-inorganic sol-gel, PHOIS, resin.

The step of forming the microfluidic channel from photolithography on a PHOIS resin may comprise forming a plurality of microfluidic channels comprising multiple y-branch microfluidic channel splitters on the PHOIS resin.

The step of forming the sensor from photolithography on a PHOIS resin may comprise forming a plurality of sensor windows on the PHOIS resin.

The method may further comprise preparing the PHOIS resin from an individual organosilane.

The method may further comprise preparing the PHOIS resin from a combination of multiple organosilanes.

The organosilanes may be selected from the group comprising: 3-(trimethoxysilyi)propylmethacrylate, 3-(triethoxysilyi)propylmethacrylate, vinyltrimethoxysilane and vinyltriethoxysilane.

The method may further comprise preparing the PHOIS resin from a combination of a single organosilane and a transition metal complex.

The method may further comprise preparing the transition metal complex from one or more transition metal alkoxide precursors and one or more ligands.

The organosilanes may comprise one or more of: 3-tri methoxypropyltrimethoxysilane, 3-triethoxypropyltrimethoxysilane, 3-aminopropyltrimethoxysilane, 3-aminopropyltriethoxysilane, vinyltrimethoxysilane, vinyltriethoxysilane, 3-Glycidyloxypropyltrimethoxysilane, Trimethoxyphenylsilane, Triethoxyphenylsilane, methyltrimethoxysilane and methyltriethoxysilane.

The method may further comprise selecting the one or more transition metal alkoxide precursors from the group comprising: titanium, zirconium, niobium, tantalum and vanadium.

The method may further comprise selecting the one or more ligands from the group comprising: acrylic acid, methacrylic acid, pivalic acid, acetone, acetyl acetone, acetic acid and maleic acid.

The method may further comprise preparing the PHOIS resin from a combination of one or more organosilane precursors and one or more transition metal precursors which has been chelated with a ligand containing a methacrylic or acrylic group.

The method may further comprise the step of incorporating one or more highly reactive groups with affinity to the target biomolecules into the PHOIS resin.

The method may further comprise the step of attaching covalent silane based linker layers containing one or more highly reactive groups to the PHOIS surface.

The method may further comprise selecting the highly reactive group from the group comprising: amino, epoxy or acrylic group.

The method may further comprise incorporating a suitable photoinitiator into the PHOIS resin.

The photoinitiator may comprise 1-hydroxy-cyclohexyl-phenyl-ketone.

The step of forming the microfluidic channel and forming the sensor from photolithography on a PHOIS resin may comprise the step of performing a standard photolithograpy process on the PHOIS resin.

The step of forming the microfluidic channel and forming the sensor from photolithography on a PHOIS resin may comprise the step of performing a two-photon polymerisation process, 2PP, on the PHOIS resin.

The step of forming the microfluidic channel and forming the sensor from photolithography on a PHOIS resin may comprise the step of performing a multiphoton-polymerisation process, MPP, on the PHOIS resin.

### BRIEF DESCRIPTION OF THE DRAWINGS

The invention will be more clearly understood from the following description of an embodiment thereof, given by way of example only, with reference to the accompanying drawings, in which:

FIG. 1 shows a top view of the biosensor system of the present invention;

FIG. 2 illustrates the main steps in the detection process of the biosensor system of the present invention;

FIG. 3 illustrates the main process steps for synthesising one exemplary PHOIS material which could be used in the biosensor system;

FIG. 4 illustrates one exemplary embodiment of the main process steps for manufacturing the biosensor; and

FIG. 5 shows a schematic of a biosensor fabricated in accordance with the process of FIG. 4.

### DETAILED DESCRIPTION OF THE DRAWINGS

The present invention provides a biosensor system for use in point-of-care applications which is capable of detecting and quantifying trace amounts of biomolecules with a high degree of sensitivity and selectivity. As shown in the figures, the biosensor system 1 comprises a microfluidic channel 2 for circulating biomolecules and a sensor 20 adapted to receive the microfluidic channel 2 and detect a plurality of different biomarkers from the biomolecules. The microfluidic channel 2 and the sensor 20 are formed on a photoreactive hybrid organic-inorganic sol-gel (PHOIS) resin material. The PHOIS resin material of the biosensor of the present invention is adapted to exhibit high surface energy, in order to provide for the efficient transfer of matter via capillary-driven fluid flow. In addition, it is adapted to provide photopatternability for microfabrication.

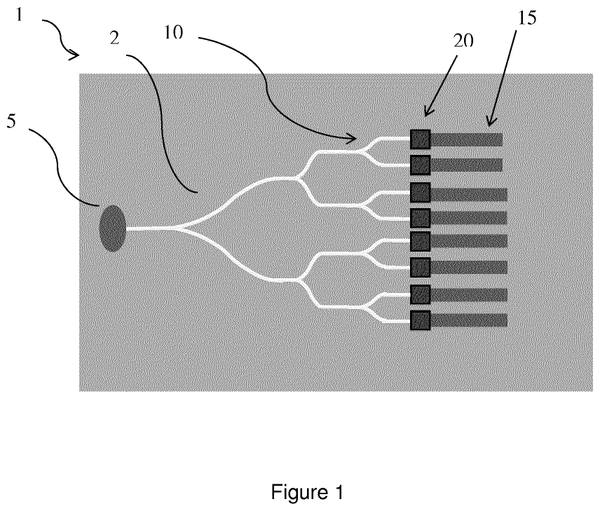

A single optical source is coupled to the sensor and adapted to excite target biomolecules to enable detection of the biomarkers by the sensor, as will be described in more detail below. In the embodiment shown in FIG. 1, the microfluidic channel comprises a plurality of microfluidic channels in the form of multiple y-branch microfluidic channels splitters 10. These multiple y-branch microfluidic channels splitters 10 are provided in order to circulate the solution of interest from an inlet 5 provided on the biosensor system into the sensor 20, which takes the form of an array of eight sensor spots or windows. This ensures an equal share of matter between the different sensor spots from a single input driving source. At the output of each sensor spot, releasing channels are patterned to circulate the liquid into a wicking zone 15.

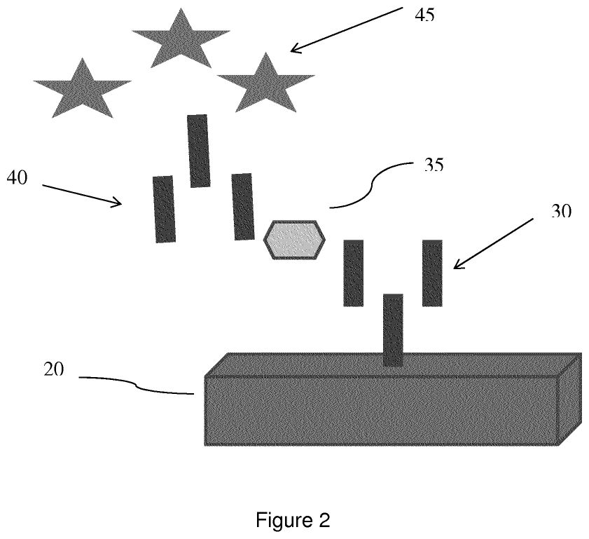

During fabrication of the biosensor of the invention, capture antibodies 30 are immobilized onto the surface of the sensor 20. Consequently, when in use, in the event that the target analyte (referred to as the antigen 35) for detection is present in the solution contained in the microfluidic channel 2, it will bind to the capture antibody 30. Subsequently, a solution of fluorescently-labelled antibodies 40 is introduced. These labelled antibodies 40 bind to any antigens 35 on the surface of the sensor 20. In order to detect the biomarkers, the fluorescently labelled surface bound antibodies are excited using visible light from the optical source. The resulting fluorescence 45 is then collected using a detector such as a CCD sensor. The detected fluorescence signal is thus related to antigen concentration, as shown in FIG. 2.

It should be noted that the antibody/antigen interaction is highly specific, with the result that the labelled antibodies will bind only to their specific antigen. This characteristic lends itself well to use of the biosensor of the present invention as a multianalyte sensor, as antibodies that are specific to different antigens can be employed on the same sensor platform.

The biosensor system of the present invention is fabricated by means of photolithography on a PHOIS resin. In one embodiment, the photolithography is the standard photolithography process (SPP), a technique widely used in the semiconductor industry. However, it will be appreciated that another photolithography process could equally well be used to fabricate the biosensor system. Examples of other processes include the two-photon polymerisation process (2PP) or the multiphoton-polymerisation process (MPP).

The PHOIS resin material of the biosensor of the present invention is one which has organic and inorganic components at a molecular level. It is synthesised via a solution-phase process known as the sol-gel process, by means of the hydrolysis and condensation of inexpensive alkoxysilane precursors. Due to the wide range of available precursors, the material is easily tailored to satisfy a large range of specifications including surface energy, functionality and curing methodologies.

The PHOIS resin can be prepared using a number of different techniques. In one embodiment of the invention, the PHOIS resin is prepared from individual organosilanes. Suitable individual organosilanes are those which contain is methacrylic or vinyl groups. These include, but are not limited to, 3-(trimethoxysilyi)propylmethacrylate, 3-(triethoxysilyi) propylmethacrylate vinyltrimethoxysilane and vinyltriethoxysilane. The PHOIS resin can alternatively be prepared from combinations of multiple organosilanes. In yet another embodiment, the PHOIS resin can be prepared from a combination of a single organosilane and a transition metal complex. In a further embodiment, the PHOIS can be prepared from the combination of single or multiple organosilane precursors and multiple transition metal precursors. The transition metal complexes can be prepared employing a single and/or multiple transition metal alkoxide precursors (such as for example titanium, zirconium, niobium, tantalum, vanadium), and a single or multiple ligands (such as for example acrylic acid, methacrylic acid, pivalic acid, acetone, acetyl acetone, acetic acid, maleic acid). Suitable precursors which can be used when combined with a transition metal that is chelated by a ligand containing a methacrylic or acrylic group (such as methacrylic acid or acrylic acid) include, but are not limited to, 3-(trimethoxysilyi)propylmethacrylate, 3-(triethoxysilyppropylmethacrylate, 3-aminopropyltrimethoxysilane, 3-aminopropyltriethoxysilane, vinyltrimethoxysilane, vinyltriethoxysilane, 3-Glycidyloxypropyltrimethoxysilane, Trimethoxyphenylsilane, Triethoxyphenylsilane, methyltrimethoxysilane, and methyltriethoxysilane.

The immobilisation of the capture antibodies to the sensor surface is required for the effective operation of the biosensor of the present invention. Antibodies are immobilised on the surface of the sensor windows using organosilane surface treatments. In order to enhance the sensitivity of the biosensor of the present invention, all biomolecular target molecules are covalently and irreversibly attached to the sensor surface. This is in contrast to most existing biosensor systems, where the immobilisation is primarily electrostatic in nature, thus resulting in limited or poor sensitivities. The present biosensor provides this uniform and efficient attachment of the biological target molecules through the use of two specific steps in the fabrication process. In the first step of the fabrication process, highly reactive groups with affinity to the target biological molecules, such as amino and/or epoxy and/or acrylic groups, are incorporated into the formulation of the PHOIS material. This is due to the fact that these chemical functionalities are able to covalently attach with the amino, hydroxyl and carboxylic acid groups which are contained in the most relevant biomolecules of interest in medical diagnostics. These biomolecules include carbohydrates, lipids, nucleic acids and proteins.

The chemical reactions which take place between the highly reactive groups and the biological molecules can be of a number of types. The first type is esterification, which is the reaction between carboxylic acids and epoxy or hydroxyl groups. The second type is amidation. This is the reaction between carboxylic acids and amino groups. The third type is polycondensation, which is the reaction between amine and epoxy groups leading to tertiary amine molecules. The fourth type is complexation of the transition metal. This is the reaction between carboxylic acids or amine groups and a transition metal complex.

In the second step of the fabrication process, covalent silane based linker layers containing amino and/or epoxy and/or acrylic groups are attached to the PHOIS surface. Such linkers are well known to preferentially create covalent bonds with biological molecules, in order to further improve the attachment of the biological target molecules to the capture antibodies. The chemical mechanism involved here are the esterification, amidation and polycondensation as previously described.

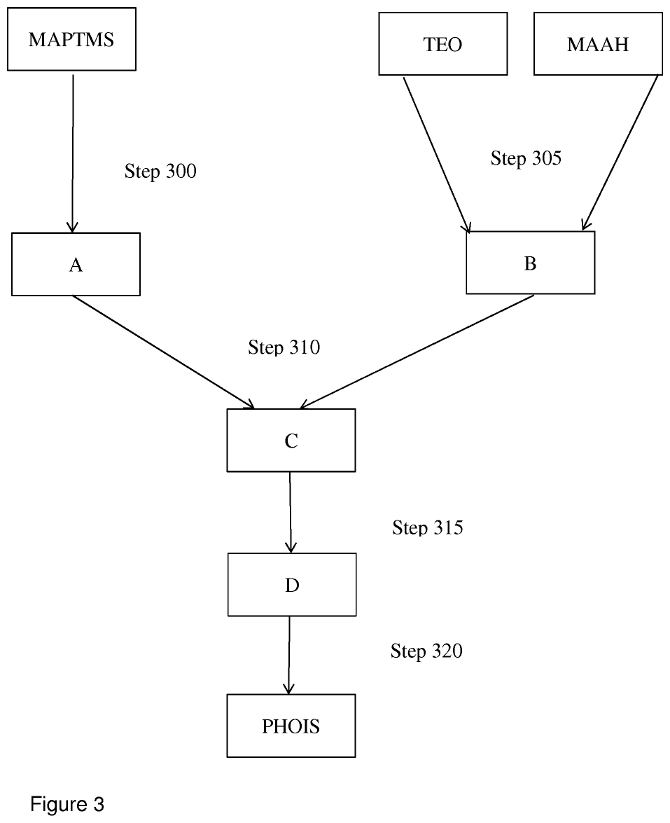

The synthesis of one exemplary PHOTS material which could be used in the biosensor of the invention will now be described in conjunction with FIG. 3. This synthesis is based on the employment of three precursors 3-(trimethoxysilyl)propylmethacrylate (MAPTMS, Assay ˜99%), tantalum (V) ethoxide (Ta(OEt)₅, also referred to as TEO), Assay ˜99.98%), and methacrylic acid (MAAH, C₄H₆O₂, Assay >98%) in a molar ratio of 2.5:1:1, respectively.

The process involves adding partially hydrolysed MAPTMS to a tantalum alkoxide complex, and then performing a further hydrolysis of the mixture. Following stirring of the resulting mixture for four hours, a suitable photoinitiator is added to create a photocurable sol-gel material. A detailed description of each step in the process is set out below.

### Step 300: MAPTMS Pre-Hydrolysis

MAPTMS is hydrolyzed with an aqueous HCl 0.1N solution in a molar ratio of 1:0.75. As MAPTMS and water are not miscible, the hydrolysis is performed in a heterogeneous way. After 5 min of stirring, the production of ethanol is sufficient to allow the miscibility of all species present in solution.

### Step 305: TEO Complexation

In parallel with the pre-hydrolysis of MAPTMS, MAAH is added dropwise to (V) TEO at a molar ratio of 1:1 in order to form a tantalum complex, where two of the reactive ethoxide groups are covalently bonded to the tantalum atom. This thus leaves the three remaining ethoxide groups available for the hydrolysis and condensation reactions with the pre-hydrolysed organosilane.

### Step 310: Addition of Pre-Hydrolysed MAPTMS to the Tantalum Complex

Following 45 minutes of stirring for both the pre-hydrolysed MAPTMS and the chelated TEO solution, the MAPTMS mixture (A) is added dropwise to the tantalum complex (B) until a 1:1 ratio of the remaining solution A to solution C is achieved. The remainder of solution A is then added directly to solution C, again, with stirring. The addition of solution A to B in this manner results in an exothermic reaction.

### Step 315: Second Hydrolysis

Following a further 45 minutes of stirring of solution C, a second hydrolysis step is performed through the addition of H₂O dropwise, such that the final hydrolysis degree is 50% against the total quantity of reactive alkoxide groups. The resulting mixture (D) is then stirred for four hours before the addition of a photoinitiator.

### Step 320: Addition of Photoinitiator

To enable the photolithographic processing of the synthesised sol-gel material, a photoinitiator is added to the final sol prior to use. In this exemplary embodiment, the photoinitiator 1-hydroxy-cyclohexyl-phenyl-ketone is used, due to its high stability in transition metal based sol-gel materials. However, it will be appreciated that different photoinitiators could equally well be used. In this exemplary embodiment, the concentration is fixed at 5 mol % (with respect to the methacrylate groups of MAPTMS). Following the addition of the photoinitiator to solution D, the resulting solution is left to stir for 1 hour. The sol is then filtered through a 0.2 μm filter prior to use.

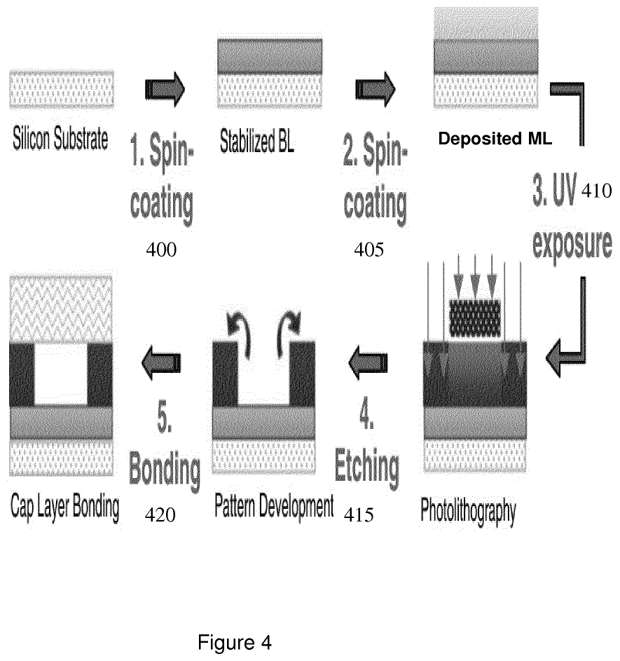

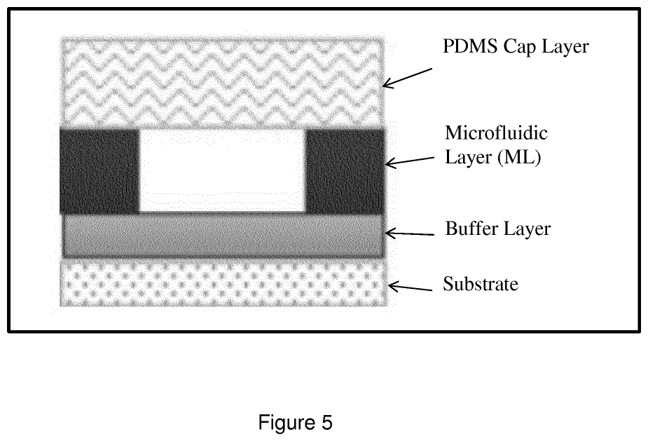

FIG. 4 illustrates one exernplary embodiment of the main process steps in the manufacture of the biosensor system of the invention, while FIG. 5 shows a schematic of a biosensor fabricated in accordance with the process.

In step 400, a silicon substrate is first coated with a ‘buffer layer’ (BL) thin film, which forms the bottom surface of the microfluidic channels. This BL is fabricated with the PHOIS resin (such as the one described with reference to FIG. 3) to facilitate the anchoring of the biomolecules in the sensor windows. This BL can be chosen based on a combination of favourable surface and processing properties. The BL is spin-coated at 1000 rpm under an alcohol saturated environment in order to achieve a coating thickness of 6 microns. Spin-coating provides a high level of film thickness control and uniformity. It is then fully stabilised by exposure to UV light for 10 min. Alternatively, the stabilisation of the BL can be performed by a thermal curing step at temperatures ranging between 100° C. and 120° C. for a duration of one hour. This stabilisation renders the BL insoluble in the alcoholic solvents used to develop the microfluidic channels in the subsequent process steps.

In step 405, the Microfluidic Layer (ML) is deposited directly onto the BL. In this embodiment, a spin-speed of 700 rpm is used, which yields a ML of 8.9 μm in height. The ML is then pre-exposure baked (PEB) on a vacuum hot-plate for 10 min at 100° C. This makes the layer sufficiently dry for the subsequent photolithography step.

Once the wafer has cooled to room temperature, the microfluidic channels are patterned in the ML. This is performed by exposure to UV light from a mask aligner through a film mask placed on the ML surface (step 410). A UV exposure time of 300 s can be used to yield a high resolution pattern. In one embodiment, low-cost emulsion film masks with 20 μm resolution can be used. In an alternative embodiment, such as for example where microfluidic channels of higher resolution are required, glass-chrome masks may be used.

In step 415, the pattern is developed by etching the UV exposed ML in 1-butanol, gently agitating the wafer in a petri dish of the solvent for 20 s, followed by rinsing with 2-propanol and then drying in a nitrogen stream. To fully stabilize the ML after exposure and etching, the wafer is then post-exposure baked for 1 hour at 110° C. in a convection oven.

Following the final bake of the PHOIS platform, a PDMS cap layer is created and bonded to the ML (step 420). To this end, a weight ratio of 1:10 of curing agent to PDMS prepolymer (SYLGARD 184 Silicone Elastomer Kit, Dow Corning, MI) are thoroughly mixed together and then degassed for 30 minutes (either under a dessicator or using sonication) to remove any bubbles in the mixture. The liquid PDMS mixture is then poured onto a silicon wafer and cured at 70° C. for 1 hour in a convection oven.

The cured PDMS cap layer is then cut and peeled from the silicon wafer surface and further divided into suitably sized pieces. Inlet and outlet points for the analytes being tested are then created in the PDMS layer. In one embodiment, a Harris Punch with a tip diameter of 3.5 mm is used to create these points.

In order to seal the biosensor, a plasma treatment step is carried out on the PDMS cap layer and the PHOIS platform. In one embodiment this is achieved by performing oxygen plasma treatment for 2 minutes using a Harrick Plasma Chamber (PDC-002), at a setting of 30 W, 0.1 Torr of O2 and a flow rate of 3 sccm. In an alternative embodiment, the plasma treatment is performed using an atmospheric plasma unit such as Openair® Plasma system. Treatment of the PDMS cap layer and the PHOIS platform also results in increased fluid flow within the microfluidic channels, due to the increased hydrophilicity of the surface following plasma treatment.

In the embodiment described above, a silicon wafer is used as a substrate. However, any other suitable substrate could equally well be used, such as for example substrates of lower cost and/or disposable substrates such as glass and PMMA, or other suitable substrates for sol-gel deposition and/or microfluidics fabrication.

It should be appreciated that the layers of sol-gel material could equally be deposited by any other suitable means in place of spin-coating, such as for example by dip-coating or spray coating.

The present invention can be used to detect biomarkers for many different applications. In one embodiment of the invention, the biosensor is used in a medical application, in respect of the detection of cardiovascular disease. This is achieved by immobilising biomolecules specific to CRP (C-reactive protein) in addition to other CVD biomarkers such as myoglobin or troponin I.

The present invention provides many advantages over the biosensor systems currently in existence. Firstly, it provides a multianalyte biosensor which is suitable for use for POC testing, due to its low cost fabrication technique. Furthermore, the highly sensitive fluorescence detection methodology incorporated into the device results in a biosensor which is simple to use by unskilled operators, such as for example a patient in their own homes. In addition, the fabrication technique involves a photolithography process, which is widely used in the semiconductor industry.

As well as having application for the detection of a broad range of diseases (such as for example cardiovascular disease), it will be appreciated that the biosensor of the present invention could also be used in other applications, such as in the detection of illicit drugs or biowarfare agents and environment pollutants.

In the specification the terms “comprise, comprises, comprised and comprising” or any variation thereof and the terms include, includes, included and including” or any variation thereof are considered to be totally interchangeable and they should all be afforded the widest possible interpretation and vice versa.

The invention is not limited to the embodiments hereinbefore described but may be varied in both construction and detail.

1. A biosensor system for use in point-of-care, POC, applications comprising:
a microfluidic channel for circulating biomolecules; and
a sensor adapted to receive the microfluidic channel and detect a plurality of different biomarkers from the biomolecules;
wherein the microfluidic channel and the sensor are formed on a photoreactive hybrid organic-inorganic sol-gel, PHOIS, resin.

2. The biosensor system of claim 1, wherein the PHOIS resin is adapted to exhibit high surface energy and photopatternability.

3. The biosensor system of claim 1, wherein the PHOIS resin is prepared from an individual organosilane, or from a combination of multiple organosilanes.

4. (canceled)

5. The biosensor system of claim 3, wherein the organosilanes comprise one or more of: 3-(trimethoxysilyl)propylmethacrylate, 3-(triethoxysilyi)propylimethacrylate, vinyltrimethoxysilane and vinyltriethoxysilane.

6. The biosensor system of claim 1, wherein the PHOIS resin is prepared from a combination of a single organosilane and a transition metal complex.

7. The biosensor system of claim 6, wherein the transition metal complex comprises one or more transition metal alkoxide precursors and one or more ligands.

8. The biosensor system of claim 7, wherein the organosilanes comprise one or more of: 3-trimethoxypropyltrimethoxysilane, 3-triethoxypropyltrimethoxysilane, 3-aminopropyltrimethoxysilane, 3-aminopropyltriethoxy silane, vinyltrimethoxysilane, vinyltriethoxysilane, 3-glycidyloxypropyltrimethoxy silane, trimethoxyphenylsilane, triethoxyphenylsilane, methyltrimethoxysilane and methyltriethoxysilane.

9. The biosensor system of claim 7, wherein the transition metal alkoxide precursors comprise one or more of: titanium, zirconium, niobium, tantalum and vanadium.

10. The biosensor system of claim 7, wherein the ligands comprise one or more of: acrylic acid, methacrylic acid, pivalic acid, acetone, acetyl acetone, acetic acid and maleic acid.

11. The biosensor system of claim 1, wherein the PHOIS resin is prepared from a combination of one or more organosilane precursors and one or more transition metal precursors which has been chelated with a ligand containing a methacrylic or acrylic group.

12. The biosensor system of claim 1, wherein one or more highly reactive groups with affinity to the target biomolecules are incorporated into the PHOIS resin.

13. The biosensor system of claim 1, wherein covalent silane based linker layers containing one or more highly reactive groups are attached to the PHOIS surface.

14. The biosensor system of claim 12, wherein the highly reactive groups comprises one or more of: amino, epoxy or acrylic group.

15. The biosensor system of claim 1, further comprising a single optical source coupled to the sensor and adapted to excite target biomolecules to enable detection of the biomarkers by the sensor.

16. The biosensor system of claim 15, wherein the sensor is adapted to detect the biomarkers through the excitation of fluorescence from the target biomolecules by means of the optical source.

17. The biosensor system of claim 1, wherein the sensor comprises a plurality of sensor windows for detection of the plurality of different biomarkers, wherein each sensor window is adapted to detect a different biomarker.

18. The biosensor system of claim 17, wherein the microfluidic channel comprises a plurality of microfluidic channels, each microfluidic channel being coupled to a different sensor window.

19. The biosensor system of claim 18, wherein one end of each microfluidic channel is coupled to an inlet for receiving the biomolecules and the other end of each microfluidic channel is coupled to its associated sensor window.

20-23. (canceled)

24. A method for fabricating a biosensor system for use in point-of-care, POC, applications, the biosensor system comprising a microfluidic channel and a sensor, the method comprising the step of:
forming the microfluidic channel and forming the sensor from photolithography of a photoreactive hybrid organic-inorganic sol-gel, PHOIS, resin.

25-38. (canceled)

39. The method of claim 24, further comprising incorporating a photoinitiator into the PHOIS resin.

40-43. (canceled)
